(19) United States
(12) Patent Application Publication
(10) Pub. No.: US 2024/0013764 A1
(43) Pub. Date: Jan. 11, 2024

(54) NOISE REDUCTION GLASSES FOR VIDEO SHOOTING

(71) Applicant: SHENZHEN YINGSHIKANG TECHNOLOGY LTD, Shenzhen, Guangdong (CN)

(72) Inventors: Qiang Guo, Shenzhen, Guangdong (CN); Yiming Wang, Shenzhen, Guangdong (CN)

(21) Appl. No.: 17/436,617

(22) PCT Filed: May 28, 2021
(86) PCT No.: PCT/CN2021/096937

§ 371 Date: Nov. 24, 2022

(30) Foreign Application Priority Data
May 17, 2021 (CN) 202121053559.2

(51) Int. Cl. G10K 11/178; H04N 23/57; H04N 23/54; H04R 1/10; G02C 5/00; G02C 11/00

(52) U.S. Cl. CPC G10K 11/17823; H04N 23/57; H04N 23/54; G10K 11/17873; H04R 1/1041; H04R 1/1083; G02C 5/003; G02C 11/10; H04R 2420/07; G10K 2210/3027; G10K 2210/1081; G10K 2210/3044; H04R 2460/01; G10K 2210/3011; G02C 2200/04

(57) ABSTRACT

The present disclosure relates to a pair of noise reduction glasses for video shooting. A frame of the glasses is provided with at least one camera unit, and an image processor is electrically connected to the camera unit and a main control unit respectively and is configured to process an image signal from the camera unit and transmit it to the main control unit; and a WiFi module is electrically connected to the main control unit and is configured to be wirelessly connected to an external terminal and transmit an image captured by the camera unit in real time. In the present disclosure, the camera unit is provided on the frame, and the WiFi module is added to transmit a real-time image, so that other users can watch a real-time scene in front of the noise reduction glasses for video shooting through a live broadcast by means of smart terminals, which meets the needs of the users for live viewing, supervision, teaching and recording, and the convenience of use is improved. In addition, the glasses of the present disclosure collect external shooting noise through a sound pickup, and then form noise-reduced audio through a sound pickup and noise reduction circuit and output it to a noise reduction earphone to realize an active shooting noise reduction function, which provide effective protection for the shooter's hearing.

### TECHNICAL FIELD

The present disclosure relates to a pair of glasses, and more specifically a pair of noise reduction glasses for video shooting.

### BACKGROUND ART

At present, safety regulations in all shooting fields require that shooters should wear both noise reduction earphones and shooting safety glasses to prevent bullet shells from flying out to damage eyes and prevent gunfire from damaging the hearing. Shooting glasses in the prior art only have a function of protecting the eyes, so when a user needs to shoot, he/she needs to wear both a pair of shooting safety glasses and a noise reduction earphone. In existing shooting glasses, a wireless connection is realized through a Bluetooth module built in the glasses to transmit audio to the earphone. However, in many cases, for example, when a shooting scene needs to be observed in real time, the existing shooting glasses cannot meet the requirements and need to be improved.

### SUMMARY

The purpose of the present disclosure is to overcome the above shortcomings of the prior art and provide a pair of noise reduction glasses for video shooting, so as to observe a real-time scene in front of the shooting glasses through a smart terminal.

To achieve the above-mentioned purpose, the technical solution used by the present disclosure is as follows: a pair of noise reduction glasses for video shooting, wherein the glasses comprise a frame on which at least one camera unit is provided, and the glasses comprise a main control unit, an image processor and a WiFi module, wherein the image processor is electrically connected to the camera unit and the main control unit respectively and is configured to process an image signal from the camera unit and transmit it to the main control unit; and the WiFi module is electrically connected to the main control unit and is configured to be wirelessly connected to an external terminal and transmit an image captured by the camera unit in real time.

The camera unit is provided in a detachable and independent piece, and the independent piece is mounted on the frame. The independent piece is fixed on a recess in a middle position of the frame in snap fit. The camera unit realizes transmission of electric energy and data through a metal contact point of the independent piece and a metal contact sheet on the frame. Or, the camera unit realizes transmission of electric energy and data through a metal contact sheet of the independent piece and a metal contact point on the frame.

The camera unit is embedded in a middle position of the frame.

The glasses further comprise a noise reduction earphone and an audio processor, and the audio processor is electrically connected to the noise reduction earphone and the main control unit respectively.

The glasses further comprise a Bluetooth module which is configured to be connected to an external smart terminal to wirelessly transmit audio and is electrically connected to the audio processor and the main control unit respectively. The noise reduction earphone has a microphone for conversation, and a voice and audio signal collected by the microphone for conversation is transmitted to the external smart terminal through the Bluetooth module.

The glasses further comprise a sound pickup and noise reduction circuit and a sound pickup, and the sound pickup and noise reduction circuit is electrically connected to the sound pickup, the main control unit and the noise reduction earphone respectively and is configured to output noise-reduced audio to the noise reduction earphone.

The glasses further comprise a power supply and a power supply squelch circuit, the power supply is configured to supply power to components of the glasses, and the power supply squelch circuit is configured to reduce a current sound of the noise reduction earphone and is electrically connected to the power supply and the sound pickup and noise reduction circuit respectively.

The glasses further comprise lenses and transparent display screens, the lenses are provided below the frame, and the transparent display screens are electrically connected to the main control unit through a video processor, are stacked on upper parts of inner sides of the lenses and are configured to display indication information.

Compared with the prior art, the present disclosure has the following beneficial effects: by providing a camera unit on a frame of a noise reduction glasses for video shooting, and adding a WiFi module to transmit real-time images to external terminals, a real-time scene in front of the noise reduction glasses for video shooting may be viewed through a live broadcast by means of a smart terminal, which meets the needs of other users for live viewing, supervision, teaching and recording, and the convenience of use is improved. In addition, the noise reduction glasses for video shooting of the present disclosure collect external shooting noise through a sound pickup, and then form noise-reduced audio through a sound pickup and noise reduction circuit and output it to a noise reduction earphone to realize an active shooting noise reduction function, which may avoid irreversible damage to the hearing during shooting, and provide effective protection for the shooter's hearing.

The above description is merely an overview of the technical solution of the present disclosure. In order to understand the technical means of the present disclosure more clearly, it can be implemented in accordance with the content of the specification, and in order to make the above and other purposes, features and advantages of the present disclosure more obvious and easier to understand, preferred embodiments are specifically cited below, which are described in detail as follows.

### BRIEF DESCRIPTION OF THE DRAWINGS

FIGS. 1 and 2 are three-dimensional views of a noise reduction glasses for video shooting of the present disclosure.

FIG. 3 is a schematic view showing the connection of electronic elements of a noise reduction glasses for video shooting of the present disclosure.

### DETAILED DESCRIPTION OF EMBODIMENTS

In order to make the purpose, technical solution and advantages of the present disclosure clearer, the present disclosure will be further described in detail below with reference to the accompanying drawings and specific embodiments.

The technical solution in the embodiments of the present disclosure will be clearly and completely described below in conjunction with the accompanying drawings of the embodiments of the present disclosure; and obviously, the embodiments described are merely some, rather than all, of the embodiments of the present disclosure. All other embodiments obtained by those skilled in the art based on the embodiments of the present disclosure without any creative effort shall fall within the protection scope of the present disclosure.

In the description of the present disclosure, it should be understood that the orientation or positional relationship indicated by terms “center”, “longitudinal”, “transverse”, “length”, “width”, “thickness”, “upper”, “lower”, “front”, “rear”, “left”, “right”, “vertical”, “horizontal”, “top”, “bottom”, “inside”, “outside”, “clockwise”, “counterclockwise” and the like is based on the orientation or positional relationship shown in the drawings, which is merely for the convenience of describing the present disclosure and for the simplification of the description, and not to indicate or imply that the means or element referred to must have a specific orientation, be constructed and operated in a specific orientation. Therefore, the above terms shall not be construed as a limitation of the present disclosure.

In the present disclosure, unless otherwise clearly specified and defined, the terms “mounted”, “connecting”, “connected”, “fixed” and other terms should be understood in a broad sense. For example, it may be a connection and may also be a detachable connection or integrated connection; may be a mechanical connection and may also be an electrical connection; and may be a direct connection and may also be an indirect connection through an intermediate medium, and may be communication between interiors of two elements or interaction relationship between two elements. For those of ordinary skills in the art, the specific meaning of the above-mentioned terms in the present disclosure should be construed according to specific circumstances.

In the present disclosure, unless otherwise clearly specified and defined, a first feature is “above” or “below” a second feature may include that the first and second features are in direct contact, or may include that the first and second features are not in direct contact but are in contact with another feature between them. Moreover, the first feature is “above”, “over” or “on” the second feature includes that the first feature is directly above or obliquely above the second feature, or it simply indicates that the first feature is higher in horizontal height than the second feature. The first feature is “below”, “under” or “underneath” the second feature includes that the first feature is directly below or obliquely below the second feature, or it simply indicates that the first feature is lower in horizontal height than the second feature.

In the description of this specification, descriptions with reference to terms “one embodiment”, “some embodiments”, “an example”, “a specific example”, or “some examples” etc., mean specific features, structures, materials or characteristics described in conjunction with the embodiment(s) or example(s) are included in at least one embodiment or example of the present disclosure. In this specification, the schematic representation of the above-mentioned terms should not be constructed as necessarily referring to the same embodiment or example. Moreover, the described specific features, structures, materials or characteristics may be combined in any one or more embodiments or examples in a suitable manner. In addition, those skilled in the art may combine different embodiments or examples described in this specification.

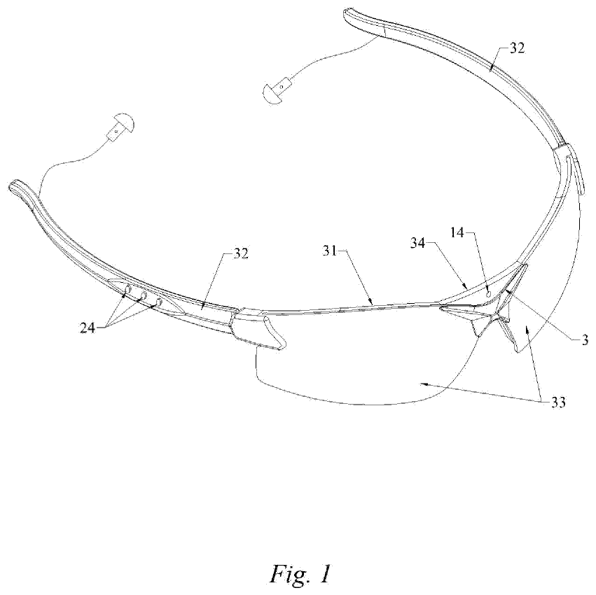

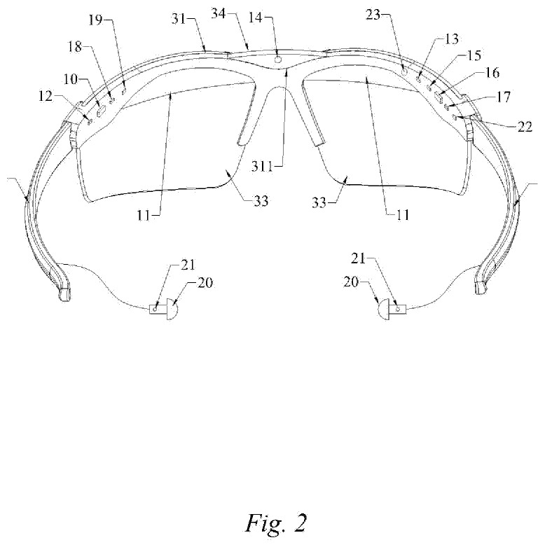

This embodiment is a pair of noise reduction glasses for video shooting. FIGS. 1 and 2 show a structural design form of the noise reduction glasses for video shooting of this embodiment, but it is not a limitation that the noise reduction glasses for video shooting of the present application must be designed according to patterns shown in FIGS. 1 and 2.

As shown in FIGS. 1 and 2, the glasses comprise a frame 31, two temples 32 and two lenses 33. The two temples 32 are respectively provided at two ends of the frame 31. The two lenses 33 are fixed below the frame 31. A camera unit 14 is provided on the frame 31. The camera unit 14 is configured to photograph a scene in front of the glasses. As shown in FIGS. 1 and 2, the camera unit 14 is provided in a detachable and independent piece 34. Such a structure may give a user the right to decide when to use the camera unit. When the camera unit is not needed, the independent piece 34 can be removed. The independent piece 34 is mounted on a recess 311 in a middle position of the frame 31, and the shape of the bottom surface of the independent piece 34 is exactly the same as the shape of the recess 311. The independent piece 34 is fixed on the recess 311 in the middle position of the frame 31 in snap fit. The independent piece 34 is provided with a tab in the middle, or a tab on each of both sides respectively. The recess 311 is provided with a bayonet/bayonets corresponding to the tab/tabs one by one, and the tab/tabs is/are aligned and inserted into the bayonet/bayonets to form a fixation.

In other embodiments, the independent piece 34 may also be fixed on the recess 311 in the middle position of the frame 31 by magnetic attraction. When in use, the independent piece 34 may be positioned and fixed automatically by being close to the recess 311, which is more convenient to use. In a specific implementation, two small magnets (not shown) may be respectively provided on the bottom surface of the independent piece 34 facing the recess 311, and iron sheets (not shown) may be provided at the corresponding positions of the recess 311 of the frame 31. Or, two iron sheets are respectively provided on the bottom surface of the independent piece 34 facing the recess 311, and small magnets are provided at the corresponding positions of the recess 311 of the frame 31. Since the independent piece 34 is symmetrical along the center position of the frame 31, the two small magnets and the two iron pieces on both sides need to be provided asymmetrically to make it have a fool-proofing function. The user can fix the independent piece 34 only when the camera unit 14 is facing forward. Of course, in other embodiments, it is also possible to provide small magnets on both the independent piece and the recess, and through the characteristics of repulsion of same poles and attraction of different poles between the small magnets, the fool-proofing is realized. In addition, in other embodiments, the outer surface of the lens of the camera unit may also be coated with an anti-fog coating to prevent the heat exhaled by the wearer of the glasses from fogging the lens and making it impossible to see the image clearly. In other embodiments, a relatively low-cost structure allows the camera unit to be directly embedded in the middle position of the frame, so that the camera unit cannot be removed.

In addition, the camera unit 14 of this embodiment realizes transmission of electric energy and data through the metal contact points (not shown) of the independent piece 34 and the metal contact sheets (not shown) on the recess 311 of the frame 31. In other embodiments, it is also possible for the camera unit to realize the transmission of electric energy and data through the metal contact sheets of the independent piece and the metal contact points on the frame.

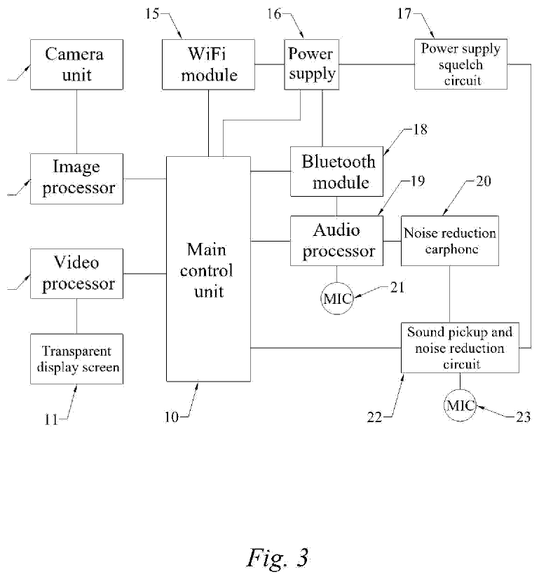

As shown in FIGS. 2 and 3, the glasses also comprise a main control unit 10, an image processor 13 (i.e. ISP), and a WiFi module 15. The image processor 13 is electrically connected to the camera unit 14 and the main control unit 10 respectively. The image processor 13 is configured to process an image signal from the camera unit 14 and transmit it to the main control unit 10. The WiFi module 15 is electrically connected to the main control unit 10, and the WiFi module 15 is configured to be wirelessly connected to an external terminal and transmit an image captured by the camera unit 14 in real time. The terminal here may be a smart terminal, such as a hand-held PDA, a smart phone, a tablet computer, or other terminals, and the terminal may also be a wireless router connected to the Internet, or the like. When the WiFi module 15 is directly connected to the smart terminal, the real-time video can be watched only in a single smart terminal. When the WiFi module 15 is connected to an Internet platform through the terminal, the real-time video captured by the camera unit 14 of the glasses will be directly transmitted to the platform for live broadcasting in real time. Other users may watch the real-time live video captured by the camera unit 14 of the glasses if a corresponding APP is installed in the smart terminals of the other users.

In other embodiments, if the main control unit is highly integrated, the image processor may also be directly integrated into the main control unit.

As shown in FIGS. 2 and 3, the glasses also comprise a noise reduction earphone 20, a Bluetooth module 18 and an audio processor 19. The audio processor 19 is electrically connected to the noise reduction earphone 20 and the main control unit 10 respectively. The noise reduction earphone 20 may be an in-ear noise reduction earphone. The Bluetooth module 18 is configured to be connected to an external smart terminal to wirelessly transmit audio. The smart terminals may be a hand-held PDA, a smart phone, a tablet computer, a smart watch, or other terminals. The Bluetooth module 18 is electrically connected to the audio processor 19 and the main control unit 10 respectively. The noise reduction earphone 20 itself has a microphone for conversation 21 which is electrically connected to the audio processor 19. A voice and audio signal collected by the microphone for conversation 21 is transmitted to the external smart terminal through the Bluetooth module 18, so that the glasses may communicate with the smart terminal in real time, which is convenient for the user to communicate with external personnel (such as guardians or coaches) in real time during shooting.

As shown in FIGS. 2 and 3, the glasses also comprise a sound pickup and noise reduction circuit 22 and a sound pickup 23. The sound pickup 23 may be provided on the frame 31. The number of the sound pickups 23 may be set to more than one. The sound pickup and noise reduction circuit 22 is electrically connected to the sound pickup 23, the main control unit 10 and the noise reduction earphone 20, respectively. The sound pickup 23 is configured to collect external noise (mainly high-decibel noise that is harmful to the hearing during shooting), and then transmit it to the sound pickup and noise reduction circuit 22 to form noise-reduced audio of an opposite phase, and finally the sound pickup and noise reduction circuit 22 outputs the noise-reduced audio the noise reduction earphone 20 to realize the active shooting noise reduction function. The noise reduction glasses for video shooting of this embodiment may protect the eyes and have the shooting noise reduction function, avoid possible irreversible damage to the eyes or the hearing during shooting, and provide effective protection for the eyes and the hearing. In addition, as shown in FIGS. 2 and 3, the glasses also comprise a power supply 16 and a power supply squelch circuit 17. The power supply 16 is configured to supply power to all components of the glasses. The power supply squelch circuit 17 is electrically connected to the power supply 16 and the sound pickup and noise reduction circuit 22 respectively. The power supply squelch circuit 22 is configured to reduce a current sound of the noise reduction earphone 20 to prevent the current sound from interfering with the user's shooting. In addition, the glasses will also be provided with a data power supply interface (not shown). The data power supply interface may be a Micro-USB interface or a Type-C interface. The data power supply interface may be configured to charge the power supply 16 or update the control software for the components of the glasses.

In the glasses structure shown in FIG. 2, most of the components are mounted in the frame 31, but in other embodiments, the temples may also be made hollow and the components are provided in the temples.

As shown in FIGS. 2 and 3, the glasses also comprise transparent display screens 11. The transparent display screens 11 are electrically connected to the main control unit 10 through a video processor 12. The transparent display screens 11 are configured to display indication information, such as remaining power, time information, shooting guidance information, and the like. The transparent display screens 11 may be LED display screens. There are two transparent display screens 11 and the two transparent display screens 11 are respectively stacked on upper parts of inner sides of the lenses 33.

Referring again to FIG. 1, several switch buttons 24 are provided on the temples 32 of the glasses. The switch buttons 24 may be provided as a general switch for the glasses, a display switch, a display brightness adjustment switch, or a sound adjustment switch, etc.

The above only uses embodiments to further illustrate the technical content of the present disclosure, so as to make it easier for readers to understand, but it does not mean that the implementation of the present disclosure is limited to this. Any technical extension or re-creation made in accordance with the present disclosure is all protected by the present disclosure. The scope of protection of the present disclosure is defined the claims.

1. A pair of noise reduction and video-recording glasses for shooting, wherein the glasses comprise a frame on which at least one camera unit is provided, and the glasses comprise a main control unit, an image processor and a WiFi module, wherein the image processor is electrically connected to the camera unit and the main control unit respectively and is configured to process an image signal from the camera unit and transmit it to the main control unit; and the WiFi module is electrically connected to the main control unit and is configured to be wirelessly connected to an external terminal and transmit a video captured by the camera unit in real time.

2. The noise reduction glasses for video shooting of claim 1, wherein the camera unit is provided in a detachable and independent piece, and the independent piece is mounted on the frame.

3. The noise reduction glasses for video shooting of claim 2, wherein the independent piece is fixed on a recess in a middle position of the frame in snap fit.

4. The noise reduction glasses for video shooting of claim 2, wherein the camera unit realizes transmission of electric energy and data through a metal contact point of the independent piece and a metal contact sheet on the frame; or, the camera unit realizes transmission of electric energy and data through a metal contact sheet of the independent piece and a metal contact point on the frame.

5. The noise reduction glasses for video shooting of claim 1, wherein the camera unit is embedded in a middle position of the frame.

6. The noise reduction glasses for video shooting of claim 1, wherein the glasses further comprise a noise reduction earphone and an audio processor, and the audio processor is electrically connected to the noise reduction earphone and the main control unit respectively.

7. The noise reduction glasses for video shooting of claim 6, wherein the glasses further comprise a Bluetooth module which is configured to be connected to an external smart terminal to wirelessly transmit audio and is electrically connected to the audio processor and the main control unit respectively; and the noise reduction earphone has a microphone for conversation, and a voice and audio signal collected by the microphone for conversation is transmitted to the external smart terminal through the Bluetooth module.

8. The noise reduction glasses for video shooting of claim 6, wherein the glasses further comprise a sound pickup and noise reduction circuit and a sound pickup, and the sound pickup and noise reduction circuit is electrically connected to the sound pickup, the main control unit and the noise reduction earphone respectively and is configured to output noise-reduced audio to the noise reduction earphone.

9. The noise reduction glasses for video shooting of claim 8, wherein the glasses further comprise a power supply and a power supply squelch circuit, the power supply is configured to supply power to components of the glasses, and the power supply squelch circuit is configured to reduce a current sound of the noise reduction earphone and is electrically connected to the power supply and the sound pickup and noise reduction circuit respectively.

10. The noise reduction glasses for video shooting of claim 1, wherein the glasses further comprise lenses and transparent display screens, the lenses are provided below the frame, and the transparent display screens are electrically connected to the main control unit through a video processor, are stacked on upper parts of inner sides of the lenses and are configured to display indication information.
